(19) United States
(12) Patent Application Publication
(10) Pub. No.: US 2026/0294667 A1
(43) Pub. Date: Oct. 1, 2026

(54) Halo Gravity Traction Data Monitoring and Analysis

(71) Applicant: Texas Scottish Rite Hospital for Children, Dallas, TX (US)

(72) Inventors: Jaysson T. Brooks, Dallas, TX (US); Brad Anthony Niese, Southlake, TX (US); Jingdong Chen, McKinney, TX (US)

(21) Appl. No.: 19/631,624

(22) Filed: Mar. 27, 2026

Related U.S. Application Data
(60) Provisional application No. 63/781,015, filed on Mar. 31, 2025.

(51) Int. Cl. A61F 5/37; A61F 5/01

(52) U.S. Cl. CPC A61F 5/3707; A61F 5/3792; A61F 2005/0197

(57) ABSTRACT

Provided herein is an apparatus, kit, and method for remotely monitoring tension in a spring-based halo gravity traction (HGT) system comprising one or more load sensors configured to be connected to a spring of the spring-based HGT system; one or more conditioning units connected to the one or more load sensors to provide power and connected physically or wirelessly to the one or more load sensors to receive a data signal from the one or more load sensors; and a data logging unit connected physically or wirelessly to the one or more conditioning units to receive and record or transmit the data signal from the one or more conditioning units.

### CROSS-REFERENCE TO RELATED APPLICATIONS

This application claims priority to U.S. Provisional Application Ser. No. 63/781,015, filed Mar. 31, 2025, the entire contents of which are incorporated herein by reference.

### STATEMENT OF FEDERALLY FUNDED RESEARCH

None.

### TECHNICAL FIELD

The present disclosure relates in general to monitoring data from a traction apparatus for the treatment of scoliosis. In particular, the present invention relates to monitoring data from a halo gravity traction (HGT) apparatus.

### BACKGROUND

Without limiting the scope of the disclosure, its background is described in connection with halo gravity traction (HGT).

Traditional HGT involves a pulley system with weights applied to the end of the pulley (see FIG. 1A, prior art). As noted by Yu (Yu et al., 2020), this type of system presents problems because the weights may hit other objects or people when ambulating around a corner, and the heavy counterweights could cause a wheelchair to tip backwards on an incline. In addition, it may be difficult for some parents to lift and apply additional weights. A spring-based HGT system has been used by surgeons to treat the most difficult scoliosis cases, where few treatment options are available. The spring-based HGT system does not experience any of the problems that a pulley-based system experiences, with the benefit of also allowing dynamic motion of the patient as they load and offload the spring (see FIG. 1B, prior art).

Roye developed best practice guidelines for when to use HGT in children with spine deformities (Roye et al., 2020). Indications for HGT include any patients with spinal curves measuring greater than 90 degrees; children with significant medical comorbidities that may not be able to tolerate an immediate spinal fusion; and children with early-onset scoliosis (less than 10 years old at onset) with smaller spinal curves measuring 60 to 90 degrees in size. McIntosh developed clinical guidelines based on the long experience of spring-based HGT (McIntosh et al., 2019). Once the halo is attached to the patient's skull, most patients start with 5 to 10 pounds of traction through the spring-based system with a goal to reach 50% of their body weight. Patients typically tolerated 10-12 hours of traction either in an upright walker or wheelchair, and usually remain in traction between 4-6 weeks.

There is equipoise as to whether it is safe to send children home while in HGT as opposed to keeping them in the hospital for the duration of their time in traction. This question is important to answer because the cost of keeping children in the hospital for 4-6 weeks is often a roadblock to using this technique in institutions across the country. A reason that equipoise exists is that little is known about the actual loads transmitted to the skull and thus the spine as the child moves and goes about their daily activities. Compliance and safety are also concerns for patients who are treated on an outpatient basis. To date, only one study has been published documenting an outpatient HGT monitoring system. Frank (Frank et al., 2023) used an ARDUINO NANO® programming board and three modules connected to it: a load cell to measure the deformity generated by the traction weight against the weight of the patient, a clock to measure the times of and intervals between recordings, and a memory card to store all the data obtained. They reported their findings on five children with scoliosis in Argentina who were treated with HGT. The system they developed produced an output of traction weight over time (see FIG. 2, prior art). The traction system used was a pulley system with weights hung on the back of it, and no remote monitoring was possible with their monitoring system. They concluded that their monitoring system indeed could allow for outpatient HGT treatment, albeit no patient was monitored for longer than seven days.

Despite these advances, a need remains for better tracking, including remote tracking, of HGT use and compliance.

### SUMMARY

As embodied and broadly described herein, an aspect of the present disclosure relates to an apparatus for remotely monitoring tension in a spring-based halo gravity traction (HGT) system comprising: one or more load sensors configured to be connected to a spring of the spring-based HGT system; one or more conditioning units connected to the one or more load sensors to provide power and connected physically or wirelessly to the one or more load sensors to receive a data signal from the one or more load sensors; and a data logging unit connected physically or wirelessly to the one or more conditioning units to receive and record or transmit the data signal from the one or more conditioning units. In one aspect, the apparatus is adapted for use with an ambulatory HGT frame. In another aspect, the apparatus is adapted for use with a wheelchair HGT frame. In another aspect, the apparatus further comprises a load sensor power source connected to the one or more load sensors. In another aspect, the apparatus further comprises a data logging power source connected to the data logging unit. In another aspect, the apparatus further comprises a conditioner power source connected to the one or more conditioning units. In another aspect, the data logging unit is configured to transmit using a wireless connection. In another aspect, the data logging unit is configured to transmit using a cellular connection.

As embodied and broadly described herein, an aspect of the present disclosure relates to a kit for an apparatus for remotely monitoring tension in a spring-based halo gravity traction (HGT) system, the kit comprising: one or more load sensors configured to be connected to a spring of the spring-based HGT system; one or more conditioning units configured to be connected to the one or more load sensors to provide power and configured to be connected physically or wirelessly to the one or more load sensors to receive a data signal from the one or more load sensors; a data logging unit configured to be connected physically or wirelessly to the one or more conditioning units to receive and record or transmit the data signal from the one or more conditioning units; and one or more cables or wires to connect the one or more load sensors, the one or more conditioning units, and the data logging unit. In one aspect, the apparatus is adapted for use with an ambulatory HGT frame. In another aspect, the apparatus is adapted for use with a wheelchair HGT frame. In another aspect, the kit further comprises a load sensor power source configured to be connected to the one or more load sensors. In another aspect, the kit further comprises a data logging power source configured to be connected to the data logging unit. In another aspect, the kit further comprises a conditioner power source configured to be connected to the one or more conditioning units. In another aspect, the data logging unit is configured to transmit using a wireless connection. In another aspect, the data logging unit is configured to transmit using a cellular connection.

As embodied and broadly described herein, an aspect of the present disclosure relates to a method of remotely monitoring tension in a spring-based halo gravity traction (HGT) system comprising: providing a patient needing HGT; providing a spring-based HGT system; providing an apparatus for monitoring tension in the spring-based HGT comprising: one or more load sensors configured to be connected to a spring of the spring-based HGT system; one or more conditioning units connected to the one or more load sensors to provide power and connected physically or wirelessly to the one or more load sensors to receive a data signal from the one or more load sensors; and a data logging unit connected physically or wirelessly to the one or more conditioning units to receive and record the data signal from the one or more conditioning units; applying the spring-based HGT system to the patient; attaching the one or more load sensors to a spring of the spring-based HGT system; receiving a data signal from the one or more load sensors with the one or more load conditioning units; and recording or transmitting the data signal with the data logging unit. In another aspect, the apparatus is adapted for use with an ambulatory HGT frame. In another aspect, the apparatus is adapted for use with a wheelchair HGT frame. In another aspect, the method further comprises a load sensor power source connected to the one or more load sensors. In another aspect, the method further comprises a data logging power source connected to the data logging unit. In another aspect, the method further comprises a conditioner power source connected to the one or more conditioning units. In another aspect, the data logging unit is configured to transmit using a wireless connection. In another aspect, the data logging unit is configured to transmit using a cellular connection.

As embodied and broadly described herein, an aspect of the present disclosure relates to an integrated monitoring control unit for remotely monitoring tension in a spring-based halo gravity traction (HGT) system comprising: a high-rate traction-force sensing device, a cloud transmission device, an automated capture control device, a platform-specific data management system, and a telemetry pipeline with gap/overlap-safe buffering, wherein the telemetry pipeline is optionally under remotely configurable policies. In one aspect, the integrated monitoring control unit of further comprises at least one of: a time-windowed aggregation device that processes one or more high-rate samples into one or more representative summaries; one or more ordered staging packs identified by sequence metadata and temporal boundaries; one or more payload structures comprising at least one of: a start time, end time, interval or window definition, and scaled-integer encoded sample values; and partial buffered data captured after a termination or controlled shutdown. In another aspect, the integrated monitoring control unit of further comprises a user interface (UI) design and logic that comprises at least one of: a displaying device ID, user ID, platform, prescribed force/compliance, live load, capture state, battery/signal, and local time; and one or more UI states/prompts selected from at least one of: baseline achieved, out of tolerance, protocol acknowledged, battery low, no signal, and tare required. In another aspect, the integrated monitoring control unit of further comprises at least one of: a distance measurement tool mounted to a horizontal frame for measuring an absolute distance to a bail ring attached to a HALO ring; wherein the distance measurement tool records one or more measured distances, traction force, compliance, platform, and time data; and wherein the one or more measured distance provides as a longitudinal treatment-response parameter for at least one of: protocol evaluation, outcome tracking, or AI model training. In another aspect, the integrated monitoring control unit further comprises at least one of synchronizing the acquisition of load-cell and inertial measurement unit data; and accessing and fusing biomechanical data selected from at least one of: active traction force, impulse, posture classification, and movement dynamics. In another aspect, the integrated monitoring control unit further comprises at least one of: one or more mobile client paired devices for remote control of the integrated monitoring control unit; one or more patient-facing and provider-facing control mode displays; and protocol upload, monitoring, and versioned change tracking through a mobile interface. In another aspect, the integrated monitoring control unit further comprises at least one of: data collection for training a machine learning module, wherein the machine learning module extracts one or more traction signals from hourly/daily/weekly/monthly monitoring data and clinical data.

As embodied and broadly described herein, an aspect of the present disclosure relates to a method of spring-based halo gravity traction (HGT) monitoring system comprising: using a processor to obtain data comprising, high-frequency traction-force acquisition, automated capture and compliance tracking, structured data aggregation and transmission, and systematic analysis of collected force and usage data to support protocol evaluation and optimization. In one aspect, the integrated monitoring control unit of further comprises at least one of: a time-windowed aggregation device that processes one or more high-rate samples into one or more representative summaries; one or more ordered staging packs identified by sequence metadata and temporal boundaries; one or more payload structures comprising at least one of: a start time, end time, interval or window definition, and scaled-integer encoded sample values; and partial buffered data captured after a termination or controlled shutdown. In another aspect, the integrated monitoring control unit of further comprises a user interface (UI) design and logic that comprises at least one of: a displaying device ID, user ID, platform, prescribed force/compliance, live load, capture state, battery/signal, and local time; and one or more UI states/prompts selected from at least one of: baseline achieved, out of tolerance, protocol acknowledged, battery low, no signal, and tare required. In another aspect, the integrated monitoring control unit of further comprises at least one of: a distance measurement tool mounted to a horizontal frame for measuring an absolute distance to a bail ring attached to a HALO ring; wherein the distance measurement tool records one or more measured distances, traction force, compliance, platform, and time data; and wherein the one or more measured distance provides as a longitudinal treatment-response parameter for at least one of: protocol evaluation, outcome tracking, or AI model training. In another aspect, the method further comprises at least one of synchronizing the acquisition of load-cell and inertial measurement unit data; and accessing and fusing biomechanical data selected from at least one of: active traction force, impulse, posture classification, and movement dynamics. In another aspect, the integrated monitoring control unit further comprises at least one of: one or more mobile client paired devices for remote control of the integrated monitoring control unit; one or more patient-facing and provider-facing control mode displays; and protocol upload, monitoring, and versioned change tracking through a mobile interface. In another aspect, the integrated monitoring control unit further comprises at least one of: data collection for training a machine learning module, wherein the machine learning module extracts one or more traction signals from hourly/daily/weekly/monthly monitoring data and clinical data.

As embodied and broadly described herein, an aspect of the present disclosure relates to a non-transitory computer readable medium for remotely monitoring tension in a spring-based halo gravity traction (HGT) system, comprising instructions stored thereon, that when executed by a computer having a communications interface, one or more databases and one or more processors communicably coupled to the interface and one or more databases, perform the steps comprising: using the processor to obtain data comprising, high-frequency traction-force acquisition, automated capture and compliance tracking, structured data aggregation and transmission, and systematic analysis of collected force and usage data to support protocol evaluation and optimization.

As embodied and broadly described herein, an aspect of the present disclosure relates to a computer-readable storage medium comprising instructions stored thereon that, in response to execution by a processor, for remotely monitoring tension in a spring-based halo gravity traction (HGT) system, comprising: obtaining, or having obtained, and storing one or more data streams from the (HGT) system; a processor in communication with the (HGT) system that: remotely monitoring tension in a spring-based halo gravity traction (HGT) system, and that using the processor to obtain data comprising high-frequency traction-force acquisition, automated capture and compliance tracking, structured data aggregation and transmission, and systematic analysis of collected force and usage data to support protocol evaluation and optimization.

### BRIEF DESCRIPTION OF THE DRAWINGS

For a more complete understanding of the features and advantages of the present disclosure, reference is now made to the detailed description of the disclosure along with the accompanying figures and in which:

FIG. 1A shows a prior-art, traditional wheelchair pulley-based HGT system. FIG. 1B shows a prior-art, traditional wheelchair spring-based HGT system.

FIG. 2 shows a graph of an output of traction weight over time for a prior-art pully-based HGT system.

FIGS. 3A to 3D show a diagram and views of an apparatus embodiment of the present invention. FIG. 3B shows an image of a specific example implementation of the apparatus embodiment of FIG. 3A. FIG. 3C shows another image of elements of the specific example implementation of FIG. 3B. FIG. 3D shows another configuration of the apparatus 300 in which the battery and controller or processor are attached to the frame, with a fixed or movable display.

FIG. 4 shows a method implementation of the present invention.

FIG. 5 shows an overview of the integrated control unit and the data transmission.

FIG. 6 shows an overview of the control and data flow

FIG. 7 shows an overview of a data acquisition architecture.

FIG. 8 shows an example of the user interface (UI).

FIG. 9 shows an example of a string style measurement tool.

FIG. 10 shows an example of a wireless MCU with IMU sensing unit mounted on the HALO ring.

FIG. 11 shows an example of a system designed to support with an end-to-end data pipeline for AI-based analysis and protocol optimization.

FIG. 12 shows an example of a methodology of data collection for AI training.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

The present disclosure includes an integrated sensing/telemetry/policy architecture that enables research-grade capture, protocol learning, and safe personalization on a battery-powered, remotely managed device. The practical value is to improve the correction rate and/or shorten treatment by delivering actionable tools and data to health providers. For example, the technology enables the study of optimization of: protocol: initial load, increment size, increment interval, daily maxima, pause/resume rules, effective interaction mode: effective dynamic traction force range and effective duration, etc.

The research-grade sensing apparatus for HGT includes, e.g., a compact load-cell transducer; an embedded controller configured for high-rate sampling and on-device aggregation; a local display providing status and a graphical user interface (GUI); multi-bearer network interfaces (Wi-Fi, cellular, satellite is also available if this feature is needed) that upload configurable data and summaries via a robust, gap- and overlap-safe algorithm; and a power-monitoring module for energy-aware operation. Optional embodiments include inertial-measurement-unit (IMU) motion sensing and closed-loop actuation (e.g., motors). Integration enables low-power deployment, scientific research (e.g., learning impacts of dynamic behavior and active force on correction rate), protocol discovery and optimization, per-patient customization, fully-automatic, and intelligent care.

The present disclosure allows for the capture of high-rate force data with 1 Hz summaries covers the necessary bandwidth of dynamic impacts that can be used to learn the optimal and most effective dynamic behavior for patients and relate it to treatment outcomes.

Static force plus the corresponding traction distance can be used to: track daily correction while reducing reliance on frequent X-rays; and can be later combined with X-ray data to derive correlations for future AI-based implementation of intelligent care.

Patient-specific protocols. By tracking daily correction and analyzing the data, the physician can customize the magnitude of traction load, dynamic traction range, effective interaction pattern, schedules of traction load, increments, timing, and alerts.

An AI-powered pipeline for decision support. Once the effectiveness of the magnitude/pattern of traction load and the corresponding dynamic frequency is captured, the system provides instructions, reminders, and commitment monitoring. Additionally, once a trend in the correspondence between X-ray outcomes and the protocol is built, the system can intelligently suggest the optimal and adaptive protocol for each patient.

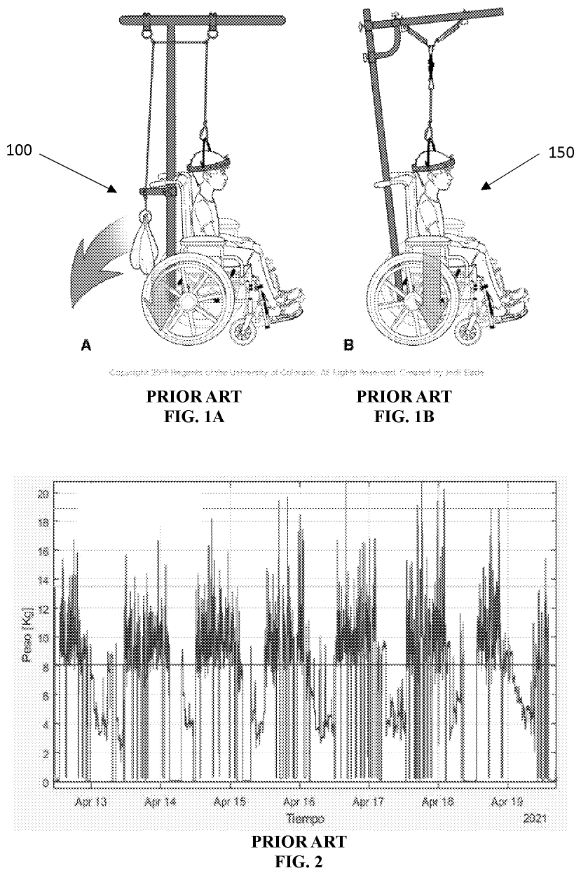

FIG. 1A shows a prior-art, traditional wheelchair pulley-based HGT system. FIG. 1B shows a prior-art, traditional wheelchair spring-based HGT system.

FIG. 2 shows a prior-art graph of an output of traction weight over time for the prior-art pully-based HGT system described by Frank et. al (Frank et al., 2023) and discussed above.

Embodiments of the present invention provide an integrated sensing/telemetry/policy architecture that enables research-grade capture, protocol learning, and safe personalization on a battery-powered, remotely managed device. The practical value is to improve correction rate or to shorten treatment by delivering actionable tools and data to health provider. For example, the technology enables the study of optimization of (1) protocol values for at least initial load, increment size, increment interval, daily maxima, and pause and resume rules, and (2) effective interaction mode parameters for at least effective dynamic traction force range and effective duration.

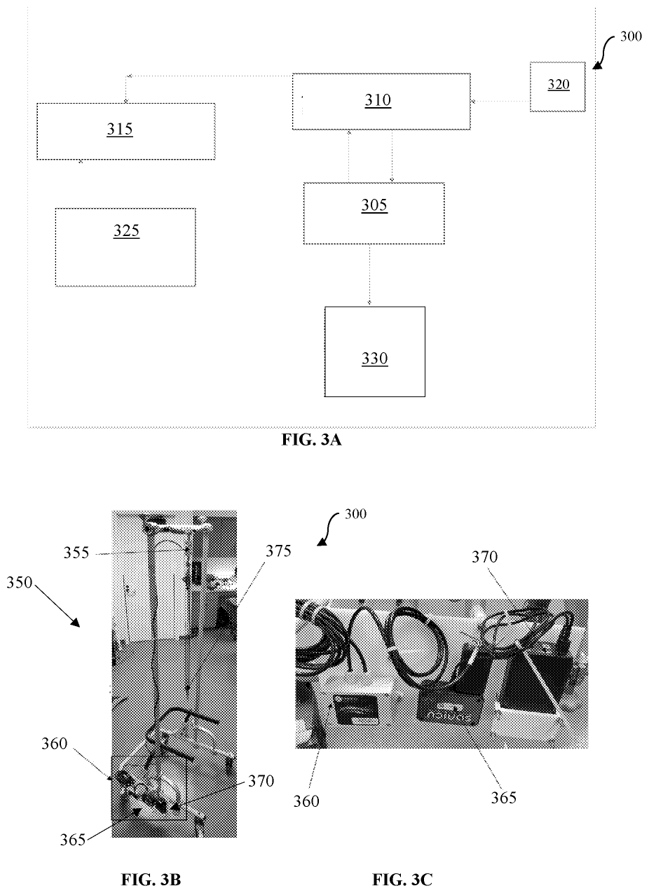

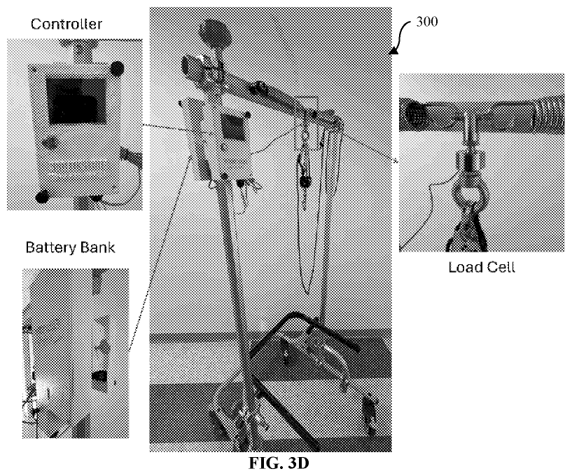

FIG. 3A shows an embodiment of the present invention, an apparatus 300 for remotely monitoring tension in a spring-based HGT system. Apparatus 300 includes one or more load sensors 305 configured to be connected to a spring of the spring-based HGT system (not shown), where the spring is to be bridged by the one or more load sensors 305. Apparatus 300 also includes one or more conditioners 310 (1) connected to the one or more load sensors 305 to provide power and (2) connected physically or wirelessly to the one or more load sensors 305 to receive a data signal from the one or more load sensors 305. Apparatus 300 further includes a data logging unit 315 connected physically or wirelessly to the one or more conditioning units 310 to receive and record or transmit the data signal (or both) from the one or more conditioning units 310. Transmission of the data signal can be performed by, e.g., a Wi-Fi™ connection or a cellular connection, and the data can be transmitted to, e.g., a remote monitoring station (not shown) or a remote storage device such as cloud storage (not shown). Also shown are a load sensor power source 320 connected to the one or more load sensors 305, a data logging power source 325 connected to the data logging unit 315, and a patient 330. Apparatus 300, and all apparatus embodiments of the present invention, are usable with wheelchair spring-based HGT systems and with walking-frame spring-based HGT systems.

FIG. 3B shows a specific example implementation 350 of the apparatus 300. This specific example implementation 350 is only an example; the present invention encompasses other apparatus implementations not discussed or shown specifically herein. The specific example implementation 350 includes a load cell 355 including RAS1-100S-A1K-T load sensors from Loadstar Sensors, which can have a 200 kg capacity and can measure with an accuracy of, e.g., 0.1, 0.5, 1, or 2 kg; an AI-1000 load conditioner 360 from Loadstar Sensors, connected to the load sensors 355; and a DVS01 data logging unit 365 from Cordova Flow Controls, connected to the load conditioner 360. FIG. 3B also shows a load cell and data logging power source 370, a 12V DC, 6000 mA power supply from TalentCell. A conditioner power source, a 9V DC battery powering the load conditioning unit 360, is not shown. FIG. 3B further shows a tensile traction load 375.

FIG. 3C shows an image of the load conditioner 360, the data logging unit 365, and the load cell and conditioner power source 370. FIG. 3D shows another configuration of the apparatus 300 in which the battery and controller or processor are attached to the frame, with a fixed or movable display.

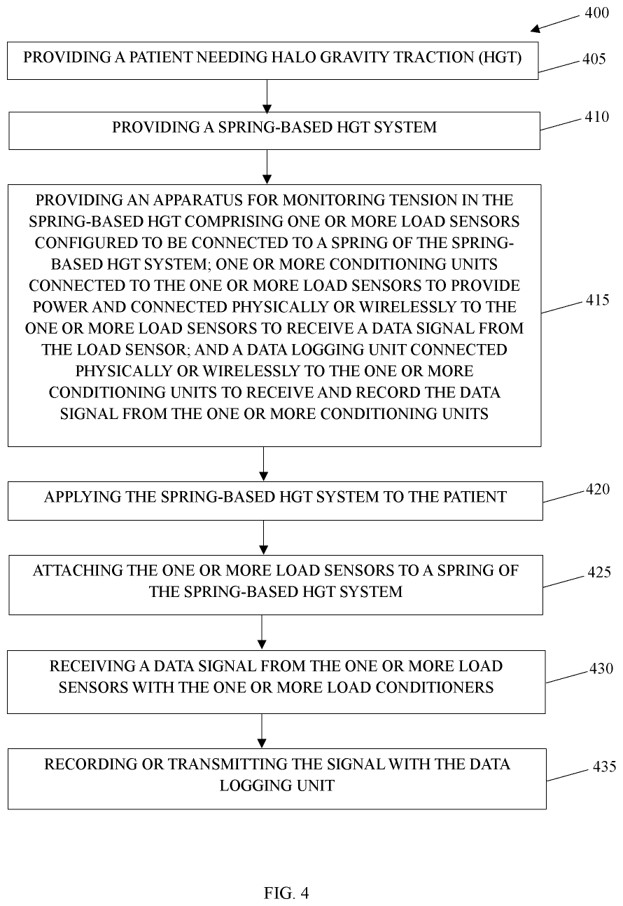

FIG. 4 shows a method implementation of the present invention. Method 400 is a method of remotely monitoring tension in a spring-based HGT system. Method 400 includes block 405, providing a patient needing HGT, and block 410, providing a spring-based HGT system. Block 415 includes providing an apparatus for monitoring tension in the spring-based HGT comprising one or more load sensors configured to be connected to a spring of the spring-based HGT system; one or more conditioning units connected to the one or more load sensors to provide power and connected physically or wirelessly to the one or more load sensors to receive a data signal from the load sensor; and a data logging unit connected physically or wirelessly to the one or more conditioning units to receive and record the data signal from the one or more conditioning units. Block 420 includes applying the spring-based HGT system to the patient. Block 425 includes attaching the one or more load sensors to a spring of the spring-based HGT system. Block 430 includes receiving a data signal from the one or more load sensors with the one or more load conditioners. Block 435 includes recording or transmitting the signal with the data logging unit. Method 400, and all method embodiments of the present invention, are usable with wheelchair spring-based HGT systems and with walking-frame spring-based HGT systems.

The present invention was used in a study of actual loads transmitted to the skull and thus the spine by a spring-based HGT system as the child that is subject to traction moves and goes about their daily activities. The purpose of the study was to develop a system and method that allows clinicians to remotely measure the dynamic forces being applied to the head and thus spine in children undergoing HGT. The primary study demonstrates the forces applied to the spine can be measured remotely on children undergoing inpatient HGT for their spine deformities. Another goal was a quantification of the hourly and daily dynamic forces applied to the spine in children undergoing outpatient HGT. Patients who are prescribed HGT treatment by their physician were approached for enrollment. Patients were enrolled prior to any HGT frame modification and were followed for the duration of their HGT treatment until discontinued. Data was collected from patients during the duration of their enrollment.

Four HGT units (two ambulatory frames and two wheelchair frames) were instrumented with the present invention, e.g., apparatus 300. The course of treatment of patients was not to be altered, nor will the additional equipment be burdensome to patients or caretakers. Additional patients can be enrolled and monitored for feasibility of the present invention for remotely measuring forces applied to the spine on children undergoing HGT. After that, additional patients are enrolled and monitored using the equipment to quantify the hourly and daily dynamic forces applied to the spine in children undergoing outpatient HGT. Each patient remains in the study from enrollment until their HGT treatment is complete and the halo has been removed from their skull. This time frame can range from a four-to-six week minimum to one year based on the treating surgeon's desired treatment regimen. Patient withdrawal is possible if patients request to discontinue study participation.

The data can be used to evaluate patient compliance and force-time integrals (testing the dynamic forces experienced in spring-based HGT). Patient compliance is evaluated by measuring the amount of time the patient is under traction over a period of time. Force-time integral is important in assessing effectivity: the general hypothesis is that higher loads and longer time periods result in better outcomes. Finally, patients are generally encouraged to be as active as possible during HGT to maintain healthy lung function and bone growth. Dynamic load data is expected to vary significantly during periods of activity and remain relatively stable during sedentary use.

Four patients requiring HGT, all female, were consecutively enrolled between June and July 2024. Patients were aged 6, 10, 12, and 13 years old, and diagnoses were infantile idiopathic, syndromic, juvenile idiopathic, and congenital, respectively. For each patient, an embodiment of the present invention was added to their halo walker and wheelchair. Traction compliance was measurable in all patients during their inpatient HGT treatment. Example force data for Patients No. 1, No. 2, No. 3, and No. 4 during the entire traction period and each minute during a 24-hour period is displayed in Tables 1, 2, 3, and 4, respectively. All patients achieved their goal traction prior to definitive fusion or growing rod instrumentation. The mean curve magnitude pre-HGT was 104 (range 83-120) degrees and at completion of HGT was 78 (range 63 to 87) degrees.

TABLE 1

| | Patient 1 | |
|---|---|---|
| Day | Standing | |
| 1 | 2.6 | (2.6-2.6) |
| 2 | 11.98 | (5.6-30.4) |
| 3 | 14.27 | (3.6-41.9) |
| 4 | 15.57 | (2-44.2) |
| 5 | 18.5 | (3.2-42.1) |
| 6 | 20.84 | (1.2-45.3) |
| 7 | 17.97 | (1-45.5) |
| 8 | 19.45 | (1.8-45.6) |
| 9 | 21.3 | (4.9-38.8) |
| 10 | 23.51 | (1.7-51) |
| 11 | 21.47 | (1-43.3) |
| 12 | 20.6 | (3.5-43.9) |
| 13 | 26.61 | (10.8-45.3) |
| 14 | 23.2 | (2.2-45.4) |
| 15 | 25.16 | (11.5-41.6) |
| 16 | 21.01 | (1-51.3) |
| 17 | 21.27 | (1.2-51.5) |
| 18 | 20.67 | (1.3-45.6) |
| 19 | 18.47 | (1.6-46.9) |
| 20 | 22.19 | (3.8-44.3) |
| 21 | 23.56 | (3.3-46.3) |
| 22 | 23.43 | (4.5-47) |
| 23 | 24.33 | (1.3-50.4) |
| 24 | 22.64 | (5.1-44.7) |
| 25 | 21.83 | (1.9-51.4) |

TABLE 2

| | Patient 2 | | |
|---|---|---|---|
| Day | | Standing | Wheelchair |

| | | | | |
|---|---|---|---|---|
| 1 | 4.12 | (1-8.2) | 17.42 | (1.1-36.2) |
| 2 | 6.44 | (1-10.1) | 15.7 | (1-32.5) |
| 3 | 9.03 | (1-13.7) | 16.13 | (1.3-34.9) |
| 4 | 12.31 | (2.4-15.6) | 17.11 | (7.3-21.7) |
| 5 | 13.6 | (2.5-20.1) | 19.15 | (2.6-32.5) |
| 6 | 12.67 | (1.8-18.5) | 17.73 | (2.4-26.4) |
| 7 | 14.23 | (2.6-18.7) | 17.43 | (5.4-30.3) |
| 8 | 17.01 | (4.9-24.6) | 12.64 | (1.9-28.5) |
| 9 | 20.79 | (5-26.8) | 15.78 | (1.3-33.6) |
| 10 | 16.31 | (5.7-26.1) | 15.53 | (7-27.8) |
| 11 | 14.26 | (3.9-20.9) | 18.44 | (6.5-29.5) |
| 12 | 20.17 | (1.3-27.7) | 18.1 | (16.8-21.3) |
| 13 | 20.67 | (1.7-33.3) | 18.23 | (3.9-23.5) |
| 14 | 19.05 | (1.1-35.9) | 15.49 | (1.6-27.3) |
| 15 | 19.31 | (1.3-33.6) | | |
| 16 | 19.09 | (5.9-53.5) | | |
| 17 | 15.71 | (1.1-42.2) | | |
| 18 | 19.76 | (1-55.2) | | |
| 19 | 20.44 | (6.3-34.8) | | |
| 20 | 20.94 | (3.3-41.3) | | |
| 21 | 21.2 | (1.2-47) | | |
| 22 | 22.18 | (1.4-59.2) | | |
| 23 | 27.07 | (11.7-51.7) | | |
| 24 | 24.68 | (6.4-55.9) | | |
| 25 | 22.83 | (1.7-45.7) | | |
| 26 | 22.85 | (1.4-57.7) | | |
| 27 | 21.46 | (1.6-43.9) | | |
| 28 | 22.45 | (1.2-46.7) | | |
| 29 | 21.81 | (8.2-55.8) | | |
| 30 | 21.15 | (3.5-46.4) | | |
| 31 | 20.18 | (10.5-38.8) | | |
| 32 | 21.94 | (7.6-44.1) | | |
| 33 | 22.98 | (4.4-61.2) | | |
| 34 | 20.13 | (2-45.1) | | |
| 35 | 22.67 | (3.7-42.3) | | |
| 36 | 19.65 | (7.4-57.9) | | |
| 37 | 21.57 | (12.1-33.2) | | |

TABLE 3

| | Patient 3 | | |
|---|---|---|---|
| Day | | Standing | Wheelchair |

| | | | | |
|---|---|---|---|---|
| 1 | 1 | (1-1) | 14.76 | (12.7-25) |
| 2 | 1 | (1-1.1) | 13.72 | (2.4-17.3) |
| 3 | 1.01 | (1-1.1) | 18.12 | (15.5-23.2) |
| 4 | 1.04 | (1-1.3) | 16.5 | (14.5-22.1) |
| 5 | 1.98 | (1-14.5) | 17.22 | (15.1-18.6) |
| 6 | 10.41 | (1-33.3) | 16.01 | (1.7-24.5) |
| 7 | 7.76 | (1-23.4) | | |
| 8 | 10.26 | (1-38.4) | | |
| 9 | 10.22 | (1-40) | | |
| 10 | 15.33 | (1-45) | | |
| 11 | 12.84 | (1-42.1) | | |
| 12 | 13.99 | (1-45.5) | | |
| 13 | 16.47 | (1-43.1) | | |
| 14 | 9.37 | (1-44) | | |
| 15 | 7.84 | (1-43.6) | | |
| 16 | 17.84 | (1.1-47.6) | | |
| 17 | 16.79 | (1-46.2) | | |
| 18 | 18.92 | (1-45.8) | | |
| 19 | 4.42 | (1-35.2) | | |
| 20 | 7.96 | (1-45.4) | | |
| 21 | 8.4 | (1-47) | | |
| 22 | 16.35 | (1.1-40.8) | | |
| 23 | 12.45 | (1.1-43.9) | | |
| 24 | 11.75 | (1-41.5) | | |
| 25 | 21.45 | (1-47.9) | | |
| 26 | 4.3 | (1-36.6) | | |
| 27 | 7.74 | (1-47.6) | | |
| 28 | 21.9 | (1.3-47.8) | | |
| 29 | 13.8 | (1.1-45.1) | | |
| 30 | 10.22 | (1.1-50.3) | | |
| 31 | 11.87 | (1.1-47.1) | | |
| 32 | 5.98 | (1.1-42.7) | | |
| 33 | 1.8 | (1.1-24.7) | | |

TABLE 4

| | Patient 4 | | |
|---|---|---|---|
| Day | | Standing | Wheelchair |

| | | | | |
|---|---|---|---|---|
| 1 | 23.79 | (2.9-60.3) | 20.14 | (1.8-28) |
| 2 | 26.25 | (1-59.5) | 17.88 | (7.6-22.3) |
| 3 | 27.33 | (10.1-58.2) | 20.43 | (3.5-27) |
| 4 | 27.81 | (16.6-59) | 19.03 | (2-23.9) |
| 5 | 31.1 | (3.5-58.7) | 24.15 | (17.2-32.3) |
| 6 | 27.6 | (17.4-57.6) | 24.28 | (6.1-31.6) |
| 7 | 27.57 | (18.6-57.7) | 24.93 | (1-33.1) |
| 8 | 30.66 | (18.6-61.6) | 26.62 | (20.5-38.6) |
| 9 | 31.62 | (6.9-56.6) | 25.74 | (19.7-31.4) |
| 10 | 27.99 | (2.5-58.1) | 26.03 | (20.2-37.5) |
| 11 | 27.47 | (16.8-57) | 26.53 | (20-35.5) |
| 12 | 31.35 | (20.2-59.3) | 28.59 | (11.1-39.6) |
| 13 | 32.62 | (18.9-58.4) | 27.51 | (22-37.3) |
| 14 | 34.18 | (21.4-58.5) | 27.03 | (2.7-35.9) |
| 15 | 28.05 | (2-57.3) | 27.79 | (22-35.3) |
| 16 | 30.7 | (14.3-58.6) | 28.89 | (1.2-37.4) |
| 17 | 26.88 | (15.6-54) | 20.19 | (1-33.8) |
| 18 | 32.97 | (8.4-58.4) | 24.94 | (18.2-33.8) |
| 19 | 33.6 | (11.3-57.7) | 27.62 | (20.8-35.7) |
| 20 | 35.23 | (22-61.7) | 28.91 | (24.5-33.8) |
| 21 | 34.71 | (10.9-59.9) | 27.89 | (20.8-34.5) |
| 22 | 33.03 | (18.3-59.6) | 29.25 | (22.8-36.8) |
| 23 | 33.03 | (1.4-60.8) | 29.48 | (25.4-37.8) |
| 24 | 34.66 | (18.2-57.6) | 31.18 | (25.6-39.3) |
| 25 | 33.11 | (21.1-60.9) | 30.12 | (23.5-44.6) |
| 26 | 32.51 | (2.7-62.3) | 27.92 | (20.7-40.4) |
| 27 | 36.16 | (24.2-59.5) | 31.38 | (26.7-36) |
| 28 | | | 31.85 | (27.1-41.6) |
| 29 | | | 29.42 | (26.8-36.4) |
| 30 | | | 29.75 | (27.2-32) |
| 31 | | | 29.5 | (26.1-34.6) |
| 32 | | | 29.71 | (24.7-38.5) |
| 33 | | | 29.04 | (6.2-41.1) |

Additionally, a cloud-managed Microcontroller Unit/Electronic Control Unit MCU/ECU platform was designed for HGT monitoring. The system acquires high-rate and accurate traction-force signals from a miniature load cell to preserve clinically relevant dynamic behavior that conventional low-frequency systems miss. The controller performs on-device processing and configurable aggregation, and transmits summarized telemetry through a structured, gap/overlap-safe data pipeline. The platform further integrates automated capture control, auto-tare of the load sensing unit, platform-specific data tracking (walker vs wheelchair), two-way cloud communication, and a modular hardware/software architecture supporting additional sensors and hardware configurations while maintaining low-power operation for long deployment periods.

Implementation of integrated IoT on HGT. The device, system and methods disclosure herein are not merely “IoT+load cell,” rather, the interaction between event aware sampling, policy tuned summarization, and energy adaptive packetization provides a field-deployable and research-grade system.

Digital real-time measurement with research-grade accuracy, enabling reliable and traceable data collection. Automated capture control and compliance monitoring improve data validity. Structured data pipeline with platform-specific data separation ensures data from different platforms (walker vs wheelchair) remains correctly attributed and analyzable. Two-way cloud communication allows device data upload and remote configuration of sampling policies, compliance targets, and system parameters.

Conventional HGT systems typically do not provide the integrated and multi-function control unit that combines high-frequency and accurate traction force sensing, automated capture control, compliance monitoring, structured and platform-specific data management, two-way cloud communication, auto-tare of load sending unit capability, informative patient-facing UI display, a structured data pipeline, and a modular hardware/software expansion within a single monitoring system.

The device includes an integrated monitoring control unit implementing high-rate traction-force sensing, cloud transmission, automated capture control, platform-specific data management, and a structured telemetry pipeline with gap/overlap-safe buffering under remotely configurable policies.

The disclosure also involves a method of HGT monitoring including high-frequency traction-force acquisition, automated capture and compliance tracking, structured data aggregation and transmission, and systematic analysis of collected force and usage data to support protocol evaluation and optimization.

The device, method, and system can include: digital, real-time load display with higher accuracy: instant display of load readings with an error of approximately ±0.1 lb, compared with the higher error associated with paper-scale measurements. Core improvement: enables accurate, research-grade data collection. Higher and more flexible sampling frequency: 1-320 Hz vs. 1/60 Hz, enabling capture of the necessary dynamic behavior and providing data to study the dynamic contribution to treatment. Automated capture on/off: improves effective data collection and allows more accurate compliance monitoring. In contrast, the version described in the patent captures data 24/7 and requires manual filtering. Structured data pipeline & platform-specific data separation: prevents data collected from the two platforms (wheelchair and walker) from being mixed together during collection and analysis.

In addition, the device, method, and system can include two-way communication capability: The cellular module can upload data to the cloud. The cloud can update environment variables and synchronize them to the controller, such as prescribed daily compliance targets and traction force.

In addition, the device, method, and system can include auto-tare for the load cell. Modular and flexible control program supports a wide range of load cells and battery configurations. Expandable controller and software design: can accommodate additional sensors, such as an Inertial Measurement Unit (IMU) motion sensor.

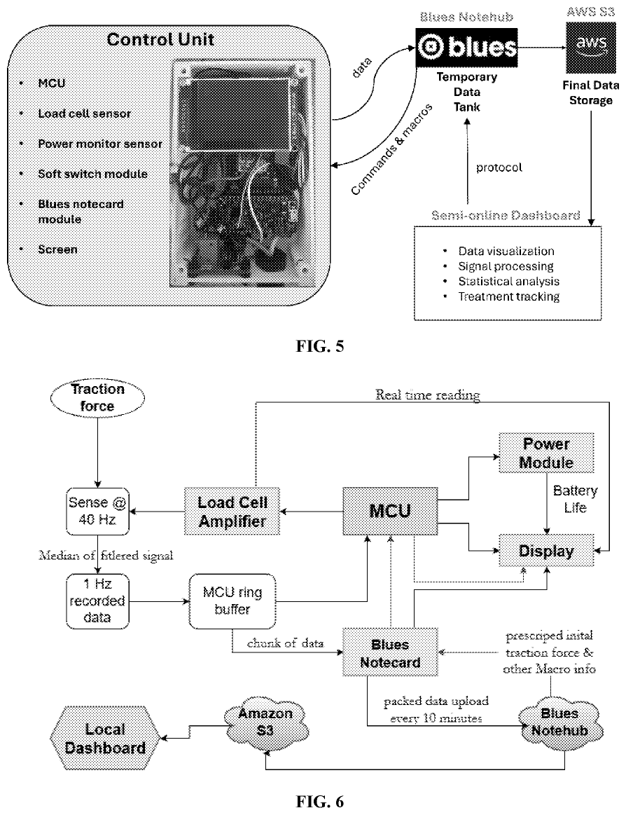

FIG. 5 shows an overview of the integrated control unit and the data transmission that includes a control unit that includes an MCU, load cell sensor, power monitor sensor, soft switch monitor, a data management module (e.g., Blues Notecard), and a screen. The control unit transmits data to the data management module, which can also transmit to a cloud server (e.g., AWS). The data management module can provide instructions and macros to the control unit. The data management module and the cloud server can include a dashboard that can include data visualization, signal processing, statistical analysis, and/or treatment tracking.

FIG. 6 shows an overview of the control and data flow. Traction force is captured and measured at, e.g., 40 Hz, and the median of the filtered signal is then recorded as data at 1 Hz. The 1 Hz data is then input into the microcontroller (MCU) ring buffer, which then transmits data to the main MCU. The MCU is connected to the load cell amplifier, which is connected to the data that is measured at 40 Hz. The main MCU is connected to the power module and a display. The display received real-time data from the load cell amplifier. A data management module (e.g., Blues Notecard) receives data from the MCU ring buffer, which it processes and transmits to the main MCU and, if applicable, the cloud. The cloud can be a data management module cloud (e.g., Blues Notecard cloud) and/or a hosting service, such as AWS. The Cloud data can then be downloaded to a local dashboard. The data management module can also deliver information to the display, and data transmitted to the data management module cloud that was uploaded, and also be processed and returned to the data management module, e.g., prescribed traction force, and other macros.

Data acquisition. Continuous high-rate sensing with on-device, time-windowed aggregation into lower-rate summaries, followed by ordered staging and persistent storage. For example, a two-stage, time-ordered buffering pipeline uses window timestamps, ordering metadata, and structured payload encoding to preserve chronological continuity, prevent duplicate upload of the same window, and support safe retransmission after communication interruption. The payload may use scaled-integer values and explicit temporal fields to reduce storage and transmission overhead.

This architecture has the advantage that it preserves short-duration load events through high-rate local acquisition while reducing bandwidth and power through on-device summarized recording. It also maintains ordered, contiguous recording across staged memory and persistent storage, reducing the risk of repeated or missing time windows during normal stop, retry, and retransmission paths. It also uses concrete metadata and payload structure, such as sequence metadata, window start/end timestamps, partial-window state, and scaled-integer sample encoding, to support compact and deterministic data handling.

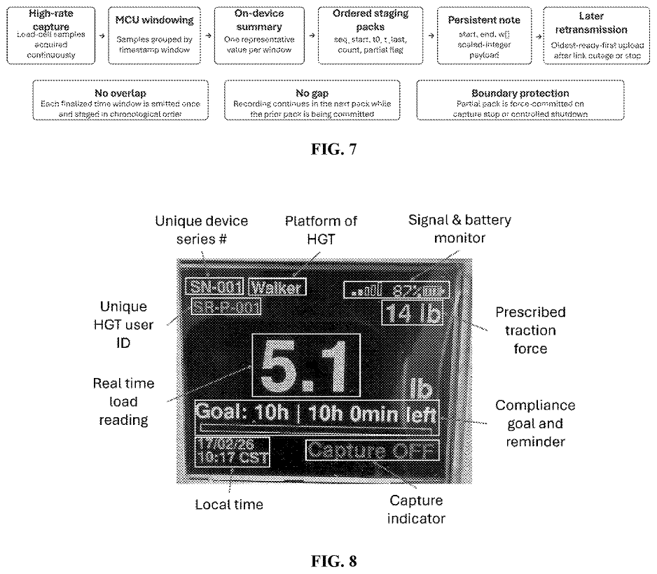

FIG. 7 shows an overview of a data acquisition architecture (where seq_start: starting sequence identifier for an ordered data pack; t0: initial timestamp of the data pack; t_last: final timestamp of the data pack; count: number of valid summarized samples in the pack; partial: indicator that the pack was force-closed before full capacity; start: persisted batch start time; end: persisted batch end time; w[ ]: array of summarized sensor values).

FIG. 8 shows an example of the user interface (UI). Local Display/GUI. The local display/GUI is not just for showing information, it is part of the system logic that helps the user operate the device correctly and helps ensure the collected data is valid, traceable, and useful for research. The design of the information shown on the UI: the unique serial number for the device; the platform: walker or wheelchair; the unique HGT user ID; signal and battery information; the prescribed traction force and compliance goal and monitoring; real-time load reading; capture on/off indicator; and/or real local time.

Advantages of the UI include: The UI is functionally tied to device operation, not just cosmetic display The UI helps the user verify the correct traction force in real time and reduces invalid data collection. The UI helps prevent baseline error, missed capture status, battery/signal issues, and protocol mismatch. The UI improves data traceability by linking the data to the correct device, user, and platform. Finally, the UI helps move the system from simple observational monitoring to more reliable, research-grade data collection.

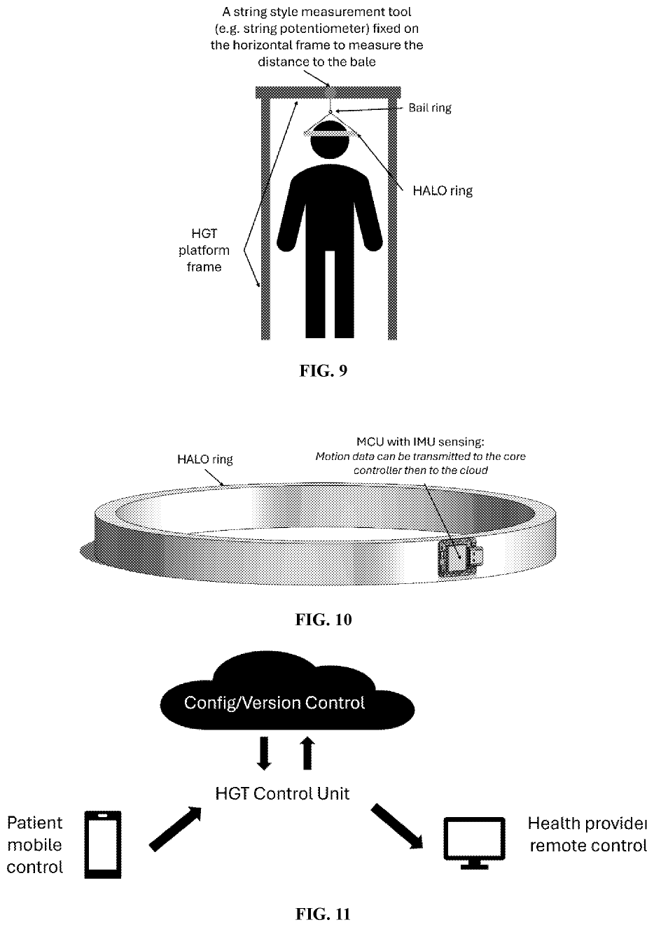

FIG. 9 shows an example of a string style measurement tool. The string style measurement tool measures absolute distance between HALO ring and frame. A frame-mounted string style distance measurement tool provides a simple and repeatable way to capture an absolute geometric treatment-response signal between the HALO ring and the wheelchair or walker frame. When recorded together with traction force, compliance, platform, and time, this distance measurement enables longitudinal progress tracking, which can reduce reliance on frequent X-ray exposure, and adds a useful input for protocol optimization, outcome prediction, and AI model training.

The design/function of the string style measurement tool. The string style measurement tool can be mounted on the horizontal frame of the wheelchair or walker platform and measures the absolute distance from the frame to the bail ring attached to the HALO ring. The string style measurement tool can be implemented as a string-style measurement tool or other compact distance measurement mechanism, records the measured distance together with traction force, time, platform, and compliance data, uses the first-day prescribed traction force as a baseline reference, and/or allows repeated distance tracking over time under comparable force conditions.

Advantages of the string style measurement tool include that it provides a practical way to collect a physical treatment-response signal without requiring frequent X-ray exposure. The string style measurement tool adds an absolute geometric measurement that can be correlated with traction protocol, compliance, and correction outcome. The string style measurement tool also enables longitudinal tracking of treatment progress under the same or similar force conditions. The string style measurement tool creates a new data stream that can improve protocol adjustment, outcome prediction, and AI model training. Finally, the string style measurement tool can be integrated with both wheelchair and walker platforms as part of the same HGT monitoring ecosystem.

FIG. 10 shows an example of a wireless MCU with IMU sensing unit mounted on the HALO ring. The sensor fusion (Inertial Measurement Unit (IMU) and load cell) is a system that integrates synchronized motion sensing (IMU) and traction force sensing (load cell) to capture both mechanical load and body movement during HGT therapy. Time-aligned signals enable the extraction of fused features such as active traction force along the spine, force impulse, motion dynamics, and posture classification. This combined sensing allows the system to analyze how patient movement interacts with traction force, providing a more complete understanding of treatment conditions and patient compliance.

Advantages of the wireless MCU with IMU sensing unit mounted on the HALO ring includes that it correlates force and motion signals, enabling higher diagnostic and analytical value compared to force-only monitoring. The wireless MCU with IMU sensing unit uses time-synchronized sensing and feature extraction allow analysis of dynamic interactions between patient posture, movement, and applied traction force. The wireless MCU with IMU sensing unit also enables new research capabilities by linking scoliosis traction mechanics with movement science and biomechanics. Finally, the wireless MCU with IMU sensing unit can also be used with conventional HGT monitoring systems that typically measure traction force alone and do not integrate synchronized motion sensing or fused biomechanical features.

FIG. 11 shows an example of a system designed to support an end-to-end data pipeline for AI-based analysis and protocol optimization. Phone/Tablet control. The system can also include a mobile client (phone/tablet) pairs with the device to provide remote control, configuration, and data access within a defined safety envelope. The mobile interface is part of the operating system logic, not just a viewer.

The design of the information shown on the UI can include: real-time force vs baseline and tolerance band; system status; progress and commitment/compliance tracking; reminders and protocol prompts; device pairing/connection status; patient-facing simplified progress view; provider-facing monitoring and configuration view; protocol authoring/upload; version history of protocol changes and updates; and/or detailed data review and analysis.

Advantages of the phone/tablet control include that the mobile UI is tied to safe device operation, not just remote display. The phone/tablet control allows for the patient and provider to have different functional roles within the same system. The phone/tablet control can also perform protocol updates, monitoring, and change history, which are integrated into one control workflow. The phone/tablet control can also be used with conventional HGT systems, which typically do not combine remote control, safety-bounded configuration, live monitoring, and versioned protocol management in one platform.

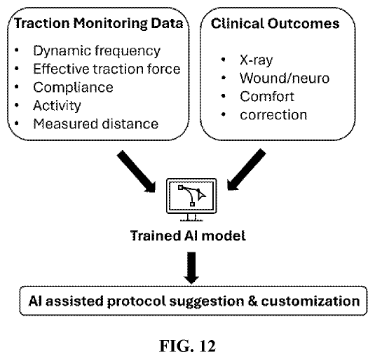

FIG. 12 shows an example of a methodology of data collection for machine learning (ML)/artificial intelligence (AI) training. The system disclosed herein is designed to support an end-to-end data pipeline for AI-based analysis and protocol optimization. The collected signals from hourly/daily/weekly/monthly dynamic/static traction, can align outcomes to the same time windows, and can use cloud-trained or device-deployed models to suggest safe protocol adjustments tailored to different patient situations.

Using the ML/AI training system, data is used to collect and train a model for optimizing treatment. The optimization can include hourly/daily/weekly/monthly summary of traction and dynamics information; static force and traction distance: the string style distance measurement along with the traction force; track patient activity; patient compliance; scheduled X-ray measurements; and/or wound status, neurological status, comfort, and respiratory function.

Advantages of the ML/AI training system include a structured pipeline that aligns traction data and clinical outcomes within matched time windows. The ML/AI training system supports extracting effective traction signals from noisy real-world usage data. The ML/AI training system links protocol performance, imaging, and patient condition into the same analysis framework. ML/AI training system model can be trained in the cloud and optionally deployed on the device for real-time decision support within a safety envelope. Finally, the ML/AI training system can also be used with conventional HGT systems, which typically do not provide this integrated data pipeline, aligned outcome mapping, and safety-bounded adaptive protocol support.

A person of skill in the art would readily recognize that steps of various above-described methods can be performed by one or more programmed computers, each having one or more computer processors. Herein, some embodiments are also intended to cover program storage devices, e.g., digital data storage media, which are machine or computer-readable and encode machine-executable or computer-executable programs of instructions, wherein the instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

A risk score of the present invention may be calculated with an algorithm using well-known statistical analysis techniques. Non-limiting examples of statistical analysis techniques that may be used to calculate the risk score include cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms. In preferred embodiments, the risk score may be calculated using a random forest algorithm using the concentrations of three or more sample analytes in the panel of biomarkers. In an exemplary embodiment, the risk score is calculated as described in the examples.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

Any and all aspects of embodiments of the present invention disclosed herein are disclosed to be present together in any single embodiment unless prevented by physical impossibility.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Frank, S., Piantoni, L., Tello, C. A., Remondino, R. G., Galaretto, E., Falconi, B. A., Pereyra, L. N., & Noël, M. A. (2023). Evaluation of outpatient halo-gravity traction in patients with severe scoliosis: development of a monitoring device. Spine Deformity, 11(2), 351-357.
2. McIntosh, A. L., Ramo, B. S., & Johnston, C. E. (2019). Halo Gravity Traction for Severe Pediatric Spinal Deformity: A Clinical Concepts Review. Spine Deformity, 7(3), 395-403.
3. Roye, B. D., Campbell, M. L., Matsumoto, H., Pahys, J. M., Welborn, M. C., Sawyer, J., Fletcher, N. D., McIntosh, A. L., Sturm, P. F., Gomez, J. A., Roye, D. P., Lenke, L. G., Vitale, M. G., & Children's Spine Study, Group. (2020). Establishing Consensus on the Best Practice Guidelines for Use of Halo Gravity Traction for Pediatric Spinal Deformity. Journal of Pediatric Orthopedics, 40(1), e42-e48.
4. Yu, H., Kim, E., & Garg, S. (2020). Development of a spring-based weight system for halo gravity traction for complex pediatric spinal deformity. Spine Deformity, 8(5), 879-884.

What is claimed is:

1. An apparatus for remotely monitoring tension in a spring-based halo gravity traction (HGT) system comprising:
one or more load sensors configured to be connected to a spring of the spring-based HGT system;
one or more conditioning units connected to the one or more load sensors to provide power and connected physically or wirelessly to the one or more load sensors to receive a data signal from the one or more load sensors; and
a data logging unit connected physically or wirelessly to the one or more conditioning units to receive and record or transmit the data signal from the one or more conditioning units.

2. The apparatus of claim 1, wherein the apparatus is adapted for use with an ambulatory HGT frame, a wheelchair HGT frame, or both.

3. The apparatus of claim 1, further comprising at least one of: a load sensor power source connected to the one or more load sensors; a data logging power source connected to the data logging unit; or a conditioner power source connected to the one or more conditioning units.

4. The apparatus of claim 1, wherein the data logging unit is configured to transmit using a wireless connection, or a cellular connection.

5. A kit for an apparatus for remotely monitoring tension in a spring-based halo gravity traction (HGT) system, the kit comprising:
one or more load sensors configured to be connected to a spring of the spring-based HGT system;
one or more conditioning units configured to be connected to the one or more load sensors to provide power and configured to be connected physically or wirelessly to the one or more load sensors to receive a data signal from the one or more load sensors;
a data logging unit configured to be connected physically or wirelessly to the one or more conditioning units to receive and record or transmit the data signal from the one or more conditioning units; and
one or more cables or wires to connect the one or more load sensors, the one or more conditioning units, and the data logging unit.

6. The kit of claim 5, wherein the apparatus is adapted for use with an ambulatory HGT frame, a wheelchair HGT frame, or both.

7. The kit of claim 5, further comprising at least one of: a load sensor power source configured to be connected to the one or more load sensors; a data logging power source configured to be connected to the data logging unit; or
a conditioner power source configured to be connected to the one or more conditioning units.

8. The kit of claim 5, wherein the data logging unit is configured to transmit using a wireless connection or a cellular connection.

9. A method of remotely monitoring tension in a spring-based halo gravity traction (HGT) system comprising:
providing a patient needing HGT;
providing a spring-based HGT system;
providing an apparatus for monitoring tension in the spring-based HGT comprising:
one or more load sensors configured to be connected to a spring of the spring-based HGT system;
one or more conditioning units connected to the one or more load sensors to provide power and connected physically or wirelessly to the one or more load sensors to receive a data signal from the one or more load sensors; and
a data logging unit connected physically or wirelessly to the one or more conditioning units to receive and record the data signal from the one or more conditioning units;
applying the spring-based HGT system to the patient;
attaching the one or more load sensors to a spring of the spring-based HGT system;
receiving a data signal from the one or more load sensors with the one or more load conditioning units; and
recording or transmitting the data signal with the data logging unit.

10. The method of claim 9, wherein the apparatus is adapted for use with an ambulatory HGT frame, a wheelchair HGT frame, or both.

11. The method of claim 9, further comprising at least one of: a load sensor power source connected to the one or more load sensors; a data logging power source connected to the data logging unit; or a conditioner power source connected to the one or more conditioning units.

12. The method of claim 9, wherein the data logging unit is configured to transmit using a wireless connection, or a cellular connection.

13. An integrated monitoring control unit for remotely monitoring tension in a spring-based halo gravity traction (HGT) system comprising:
a high-rate traction-force sensing device, a cloud transmission device, an automated capture control device, a platform-specific data management system, and a telemetry pipeline with gap/overlap-safe buffering, wherein the telemetry pipeline is optionally under remotely configurable policies.

14. The integrated monitoring control unit of claim 13, further comprising at least one of:
a time-windowed aggregation device that processes one or more high-rate samples into one or more representative summaries;
one or more ordered staging packs identified by sequence metadata and temporal boundaries;
one or more payload structures comprising at least one of: a start time, end time, interval or window definition, and scaled-integer encoded sample values; and
partial buffered data captured after a termination or controlled shutdown;
a user interface (UI) design and logic that comprises at least one of:
a displaying device ID, user ID, platform, prescribed force/compliance, live load, capture state, battery/signal, and local time; and
one or more UI states/prompts selected from at least one of: baseline achieved, out of tolerance, protocol acknowledged, battery low, no signal, and tare required;
a distance measurement tool mounted to a horizontal frame for measuring an absolute distance to a bail ring attached to a HALO ring;
wherein the distance measurement tool records one or more measured distances, traction force, compliance, platform, and time data; and
wherein the one or more measured distance provides as a longitudinal treatment-response parameter for at least one of: protocol evaluation, outcome tracking, or AI model training;
synchronizing the acquisition of load-cell and inertial measurement unit data; and
accessing and fusing biomechanical data selected from at least one of: active traction force, impulse, posture classification, and movement dynamics; or
one or more mobile client paired devices for remote control of the integrated monitoring control unit;
one or more patient-facing and provider-facing control mode displays; and
protocol upload, monitoring, and versioned change tracking through a mobile interface.

15. The integrated monitoring control unit of claim 13, further comprising at least one of: data collection for training a machine learning module, wherein the machine learning module extracts one or more traction signals from hourly/daily/weekly/monthly monitoring data and clinical data.

16. A method of spring-based halo gravity traction (HGT) monitoring system comprising:
using a processor to obtain data comprising, high-frequency traction-force acquisition, automated capture and compliance tracking, structured data aggregation and transmission, and systematic analysis of collected force and usage data to support protocol evaluation and optimization.

17. The method of claim 16, further comprising at least one of:
a time-windowed aggregation device that processes one or more high-rate samples into one or more representative summaries;
one or more ordered staging packs identified by sequence metadata and temporal boundaries;
one or more payload structures comprising at least one of: a start time, end time, interval or window definition, and scaled-integer encoded sample values; and
partial buffered data captured after a termination or controlled shutdown;
a user interface (UI) design and logic that comprises at least one of:
a displaying device ID, user ID, platform, prescribed force/compliance, live load, capture state, battery/signal, and local time; and
one or more UI states/prompts selected from at least one of: baseline achieved, out of tolerance, protocol acknowledged, battery low, no signal, and tare required;
a distance measurement tool mounted to a horizontal frame for measuring an absolute distance to a bail ring attached to a HALO ring;
wherein the distance measurement tool records one or more measured distances, traction force, compliance, platform, and time data; and
wherein the one or more measured distance provides as a longitudinal treatment-response parameter for at least one of: protocol evaluation, outcome tracking, or AI model training;
synchronizing the acquisition of load-cell and inertial measurement unit data; and
accessing and fusing biomechanical data selected from at least one of: active traction force, impulse, posture classification, and movement dynamics; or
one or more mobile client paired devices for remote control of the integrated monitoring control unit;
one or more patient-facing and provider-facing control mode displays; and
protocol upload, monitoring, and versioned change tracking through a mobile interface.

18. The method of claim 16, further comprising at least one of:
data collection for training a machine learning module, wherein the machine learning module extracts one or more traction signals from hourly/daily/weekly/monthly monitoring data and clinical data.

19. A non-transitory computer readable medium for remotely monitoring tension in a spring-based halo gravity traction (HGT) system, comprising instructions stored thereon, that when executed by a computer having a communications interface, one or more databases and one or more processors communicably coupled to the interface and one or more databases, perform the steps comprising:
using the processor to obtain data comprising, high-frequency traction-force acquisition, automated capture and compliance tracking, structured data aggregation and transmission, and systematic analysis of collected force and usage data to support protocol evaluation and optimization.

20. A computer-readable storage medium comprising instructions stored thereon that, in response to execution by a processor, for remotely monitoring tension in a spring-based halo gravity traction (HGT) system, comprising:
obtaining, or having obtained, and storing one or more data streams from the (HGT) system;
a processor in communication with the (HGT) system that:
remotely monitoring tension in a spring-based halo gravity traction (HGT) system, and that using the processor to obtain data comprising high-frequency traction-force acquisition, automated capture and compliance tracking, structured data aggregation and transmission, and systematic analysis of collected force and usage data to support protocol evaluation and optimization.
